(19) United States
(12) Patent Application Publication
(10) Pub. No.: US 2006/0003977 A1
(43) Pub. Date: Jan. 5, 2006

(54) Tibolone-adsorbates

(76) Inventor: Klaus Glaenzer, Hamburg (DE)

Correspondence Address: J C PATENTS, INC., 4 VENTURE, SUITE 250, IRVINE, CA, 92618, US

(21) Appl. No.: 11/180,728

(22) Filed: Jul. 12, 2005

Related U.S. Application Data
(63) Continuation-in-part of application No. 11/051,095, filed on Feb. 4, 2005.

(30) Foreign Application Priority Data
Dec. 22, 2004 (EP) 04 106 913.9
Feb. 6, 2004 (EP) 04 002 682.5

(51) Int. Cl. A61K 31/573

(52) U.S. Cl. 514/178

(57) ABSTRACT

The present invention relates to a process for the preparation of powders containing active ingredients in which amorphous tibolone is present in a morphologically stable form, where one starts with a solution of tibolone in at least one organic solvent where the total water content of the solvent is not higher than 15% by volume, preferably not higher than 5% by volume, dissolves therein wholly or in part a carrier material selected from the group of acrylic polymers, and removes the solvent.

### CROSS-REFERENCE TO RELATED APPLICATION

This application is a continuation-in-part of a prior application Ser. No. 11/051,095, filed Feb. 4, 2005. The prior application Ser. No. 11/051,095 claims the priority benefit of European application serial no. 04 002 682.5, filed on Feb. 6, 2004. The instant application also claims the priority benefit of European application serial no. 04 106 913.9, filed on Dec. 22, 2004. All disclosures are incorporated herewith by reference.

### BACKGROUND OF THE INVENTION

1. Field of the Invention

The present invention relates to a novel process for preparing powders containing active ingredients in which amorphous tibolone is present in a morphologically stable form. More particularly, the present invention relates to a process for preparing such tibolone-containing powders which are characterized by the fact that in them, tibolone is present in an amorphous form. The invention further relates to pharmaceutical formulations that can be prepared while employing said powder containing active ingredients in which tibolone is present. Preferred drug formulations according to the invention are tablets and granules prepared with the usual, pharmaceutically acceptable adjuvants in ways known per se. Particularly preferred according to the invention are tablets rapidly releasing the active ingredient, tibolone, which are prepared by direct compressing of the powder according to the invention which contains tibolone in an amorphous form as the active ingredient.

2. Description of the Related Art

The medicinal substance known by the INN tibolone is familiar to chemists under the synonym of 17-hydroxy-7α-methyl-19-nor-17α-pregn-5(10)-en-20-in-3-one (IUPAC) (for the formula see FIG. 1). Tibolone is a synthetic steroid resembling norethisterone and having a combined estrogenic, androgenic, and gestogenic activities. The two hydroxy metabolites (3α and 3β) (for the formulas see FIGS. 3 and 4) have an activity similar to that of estrogen, while the Δ⁴ isomer (for the formula see FIG. 2) reveals a predominantly gestogenic and androgenic activity owing to its binding to gestogen and androgen receptors (cf www.wechseljahre.com, 7α-Methyl-17α-ethinyl-estradiol (for the formula see FIG. 5), a further metabolite recently identified, has a strong estrogenic activity (cf. Steroids 67 8 (2002), 681-686). In daily doses of 2.5 mg, tibolone has therapeutic uses, amongst others, for hormone substitution in the postmenopause (see, for instance, Drugs Fut. 6, 302 (1998)).

Tibolone is an unstable substance. Under the effects of heat, acidic or oxidative interactions, the corresponding degradation processes already start during synthesis or while manufacturing the drug formulation. This is particularly pronounced in all granulation procedures or when spraying solutions of tibolone. In this way decomposition and/or isomerisation processes are induced which give rise to insufficient stability of the final product. With the intention of obtaining more stable formulations, stabilising additives with pH-controlling and antioxidant properties have been proposed according to WO 03/032924, and described in the instance of pregrinding with sodium bicarbonate or sodium citrate followed by wet granulation.

Apart from the transformation to stereoisomeric compounds in the dissolved state that has already been mentioned, the morphology obtained upon precipitation or crystallisation is another special feature of tibolone. Depending on the conditions of drying, precipitation, or crystallisation, different solids or morphologies are obtained. Depending on the solvent systems, predominantly polymorph I can for instance be obtained from selected polar solvents, predominantly polymorph II from selected nonpolar solvents, or else, their mixtures can be obtained (EP 0 359 035 A1). The process of crystallization and the phase transitions depend on a large number of factors that are difficult to control (see, for instance, S. X. M. Boerrigter et al, J. Phys. Chem. B, 4725-4731 (2002); 14^{th} Int. Symp. on Industrial Crystallization, Cambridge (1999); EP-A-389 035). It must be noted in particular that minor changes in the conditions of crystallization, in the solvent systems, in the degree of saturation, in the seed crystals and conditions of drying will strongly influence the development of a particular morphology.

It must be remarked, moreover, that tibolone in an amorphous, morphologically stable form has so far been described, neither as a substance nor as an ingredient of pharmaceutical formulations. According to findings of the inventor of the present application, this must be attributed to the very fast transformation to crystalline forms that is typical for this substance.

Apart from these morphological conditions of the active ingredient, uncertain and in part escaping an exact definition, a phase change that can occur under particular conditions during prolonged storage of the formulations constitutes a further quality problem that is difficult to control.

It must be remembered in this context that different polymorphs have a different dissolution behaviour. For instance, the tibolone crystals of form II exhibit a lower dissolution rate than those of form I. This in turn will produce different, variable dissolution profiles of the drugs manufactured from them (see EP-A-389 035). It can be concluded, therefore, that there is a very high risk of uncontrolled transformation to polymorphous or isomeric forms, most particularly in processes for the manufacture of pharmaceutical preparations in which the tibolone active ingredient must be pre-dissolved or partly dissolved, so that it is not guaranteed that regulatory requirements as to a clearly defined, active ingredient or constant quality of the drug can be met.

### SUMMARY OF THE INVENTION

Therefore, it is an object of the present invention to develop a simple and economical process for the preparation of oral tibolone formulations avoiding the disadvantages described above.

It is a further object of this invention to develop powders containing active ingredients in which amorphous tibolone is present in a morphologically stable form.

It is a further object of this invention to develop a process for obtaining these powders containing active ingredients in which amorphous tibolone is present in a morphologically stable form.

It is a further object of this invention to develop a process for preparing oral pharmaceutical formulations in the form of tablets and granules based on the powders containing tibolone that were quoted above.

According to the invention, the object is solved by a process for the preparation of powders containing amorphous tibolone according to which one starts from a solution of tibolone in at least one organic solvent, disperses and/or dissolves the carrier material in it, and then removes the solvent in a suitable way, which can more particularly be achieved by drying. According to the invention, the total water content of the solvent is not higher than 15% by volume, preferably not higher than 5% by volume (cf Claim 1). Carrier materials according to the invention are pharmaceutically acceptable acrylic acid polymers and acrylic acid copolymers. Preferred carrier materials are polymethacrylates available commercially as Eudragits® (see Fiedler, Lexikon der Hilfsstoffe, Editio Cantor publishers, 2000, pp. 698-690), particularly preferred carrier materials are aminoalkyl methacrylates.

According to the invention, the ratio between active ingredient, that is, tibolone, and carrier is set in the range from 1:0.1 to 1:10, and more particularly in the range from 1:0.5 to 1:5.

The invention further relates to pharmaceutical formulations containing these new powders containing active ingredients in which amorphous tibolone is present in a morphologically stable form. Where applicable, the pharmaceutical formulations according to the invention contain further adjuvants and can be converted to the desired drug delivery form. Tablets produced by direct compressing which rapidly release the active ingredient, and which where applicable may be coated, are particularly preferred.

### BRIEF DESCRIPTION OF THE DRAWINGS

FIG. 1 shows the structural formula of tibolone,

FIG. 2 shows the structural formula of the Δ⁴ isomer of tibolone,

FIG. 3 shows the structural formula of the 3α-hydroxy metabolite of tibolone,

FIG. 4 shows the structural formula of the 3,β-hydroxy metabolite of tibolone,

FIG. 5 shows the structural formula of 7α-methyl-17α-ethinylestradiol, a metabolite of tibolone,

FIG. 6 shows a powder X-ray diffraction pattern of an amorphous tibolone powder according to the invention, prepared according to Example No. 1,

FIG. 7 shows a powder X-ray diffraction pattern of an amorphous tibolone powder according to the invention, prepared according to Example No. 3,

FIG. 8 shows a powder X-ray diffraction pattern of an amorphous tibolone powder according to the invention, prepared according to Example No. 4,

FIG. 9 shows a powder X-ray diffraction pattern of an amorphous tibolone powder according to the invention, prepared according to Example No. 4 and stored for three months at 30° C.

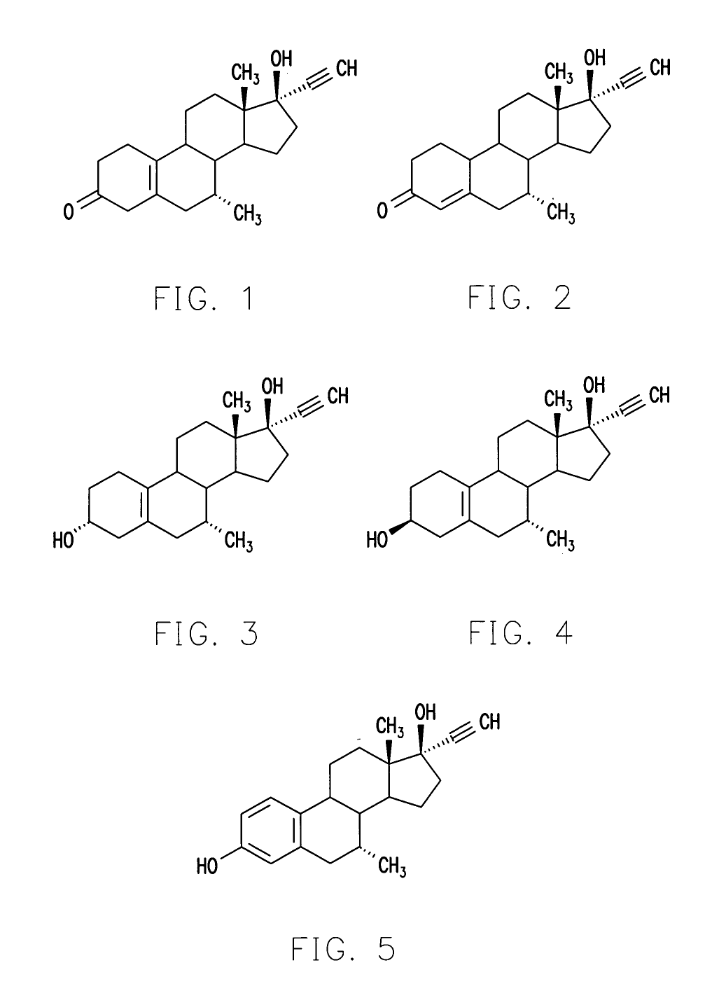

### DESCRIPTION OF THE PREFERRED EMBODIMENTS

For the process according to the present invention, of manufacturing the powders containing active ingredients in which amorphous tibolone is present in a morphologically stable form, organic solvents are suitable for the solution containing the active pharmaceutical ingredient. The organic solvents are more particularly selected from the group of lower alkanols with one to four carbon atoms, the group of ethers, the esters, the group of polyvalent alcohols such as glycol, and the group of aliphatic ketones, as well as mixtures of said solvents. Methanol, ethanol, isopropanol, n-propanol, acetone and other solvents such as ethyl acetate, methyl ethyl ketone, MTBE (methyl tert-butyl ether), dichloromethane, petroleum ether, hexane, an acetone/water mixture, an acetone/hexane mixture, an ethyl acetate/hexane mixture, dichloromethane/ethyl acetate, as well as mixtures of said solvents are particularly preferred. According to the invention, it is preferred to work under inert gas, for instance under nitrogen, and where necessary using degassed or deoxygenated solutions. Basic substances such as volatile nitrogen bases (for instance alkylamines or even pyridine) or antioxidants (for instance ascorbyl palmitate, butylhydroxytoluene, butylhydroxyanisol) can also be present as additives in the solutions.

All common pharmaceutical adjuvants can be used to prepare the pharmaceutical formulations, where tablets are more particularly preferred. As fillers and/or binders, for example, celluloses and cellulose derivatives (for instance microcrystalline cellulose, native cellulose, hydroxypropyl cellulose, hydroxypropyl methyl cellulose, methyl cellulose), sugars (for instance lactose, fructose, saccharose, glucose, maltose), sugar alcohols (for instance lactitol, mannitol, sorbitol, xylitol), inorganic fillers (for instance calcium phosphates and calcium sulfates), and starches (for instance corn starch, potato starch, wheat starch, dextrins, pregelatinized starches) can be used. Beyond that, all other adjuvants known to those skilled in the art from their basic galenic knowledge, such as lubricants, disintegration aids, wetting agents, agents to improve the flow behavior, alkaline additives, stabilizers, as well as flavors, pigments, and dyes, can be used to prepare the drug formulations according to the invention.

The portion of binders in the complete mixture of the drug preparation is preferably between 0 and 20% (m/m), the fraction of fillers and adjuvants in the complete mixture is 20 to 99% by weight, preferably 50 to 99% by weight.

With the process according to the invention, tibolone preparations are manufactured which are invariable, more particularly, with respect to their morphology, too, and which, moreover, are characterized by a rapid release of the active ingredient in water under physiological conditions. According to the invention, a process has been found which, starting from a solution of tibolone in an organic solvent, leads to powder with amorphous active ingredient that is suitable for immediate further processing.

In an embodiment of the invention, the solution of tibolone active ingredient can on principle be prepared by dissolving the tibolone in a suitable organic solvent; though it is more advantageous to directly use a solution of the active ingredient resulting anyhow during synthesis, without isolation of the tibolone.

Tibolone can for instance be prepared according to Wieland and Anner, Helv. Chim. Acta, 1453-1461 (1967), but omitting the recrystallization steps involving a dissolution in dichloromethane/ether, and instead dispersing the carrier material in the solution of the active ingredient, and later removing the solvent. The kind of organic solvent used then results, in any given case, from the final step of synthesis in the process chosen for preparing the active ingredient.

The drying process can be promoted by temperature and by applying a vacuum. Equally well, freeze drying can be used as the drying process (see examples following below). The solvent can be recovered by working in a closed system, and reused for a subsequent process. According to the invention, a precipitation and isolation of the tibolone is omitted. Preparations containing tibolone that have been prepared by the process according to the invention that has been described, can be employed directly in further processing to drug formulations such as tablets or granules, preferably in further processing by a direct compressing process.

Where necessary for specific applications, the drug formulations thus obtained can be further provided with the common film coatings, for instance for controlled release and/or taste masking and/or improved stability, as described for instance in Chapter 9 of the monograph of W. A. Ritschel, Die Tablette, Editio Cantor publishers, 2002, and the literature cited there.

It has been found surprisingly that preparations manufactured by the process according to the invention contain the active ingredient in the homogeneous distribution required for drugs, and release it without limitations. The crystal structures of the two modifications of tibolone known up to now (monoclinic and triclinic), or other crystalline forms if present, cannot be detected in the preparations according to the invention. Rather, always purely amorphous structures of tibolone are obtained.

The characteristics mentioned are retained as well, more particularly, when the tibolone preparations are processed to drug formulations, e.g., tablets. Moreover, this direct processing does not entail any change in the content of by-products or decomposition products (=sum of all contaminants) in the route from active ingredient to drug formulation (tablet).

The invention will now be explained more closely with the aid of examples, without however limiting the invention thereto.

### EXAMPLES 1 To 4

Methods of Analysis Used

1. HPLC method for determining the content of active ingredient or sum of all contaminants

All HPLC measurements were performed with an Agilent 100 HPLC.

| | |
|---|---|
| Column used: | CC 250/4 Nucleosil 100-5 C18 |
| Mobile phase: | 50% aqueous ammonium |
| | bicarbonate solution (0.005%) |
| | 50% acetonitrile |
| Flow rate: | 1.0 ml/min |
| Detector: | UV, 210 nm |
| Injection volume: | 20 μl |
| Tibolone retention time: | about 13 min |
| Analysis time: | 60 min |

2. Release of active ingredient (dissolution test) according to Ph. Eur., 1000 ml water, 0.25% sodium lauryl sulfate (required: a minimum of 80% released after 30 min)

3. The powder X-ray diffraction patterns were recorded as follows:

| | |
|---|---|
| Instrument: | STADI P transmission diffractometer |
| | Cu Kaₗ radiation (l = 1.54056Å), U = 40 kV, I = 30 mA |
| | Secondary beam monochromator (flat, graphite) |
| Detector: | Scintillation counter |
| Aperture: | 2 × 8 mm; 0.7 mm; 0.35 mm |
| Linear PSD: | 2 θ = 2° to 35°, 5 s/0.04° in steps |
| Sample: | Powder, reflection mode |

### Example No. 1

### Preparation of Tablets with Amorphous Tibolone, Formula I (According to the Invention)

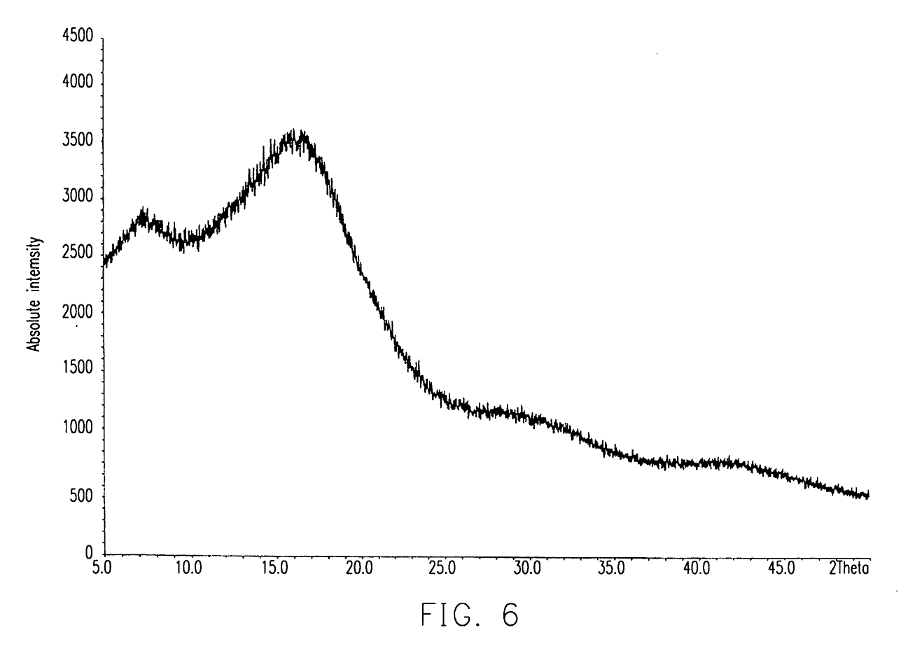

To a solution of 1 g crystalline tibolone in 16 ml acetone, 2 g polymethacrylate (Eudragit® E) are added and dissolved under gentle heating (30 to 40° C.). The transparent solution is freeze-dried so as to obtain a fine powder containing active ingredients in which amorphous tibolone is present (in the following called “tibolone powder”). The X-ray diffraction pattern of the tibolone powder is presented in FIG. 6; it reveals no crystalline fractions, only amorphous fractions. The tibolone powder is mixed with pharmaceutical adjuvants according to the following formula, and compressed to tablets:

| | |
|---|---|
| Tibolone powder corresponding to 2.5 mg tibolone | 7.5 mg |
| Microcrystalline cellulose (Celphere SCP-100 ®) | 77.5 mg |
| Adjuvants (croscarmellose sodium, sodium lauryl sulfate, | 15 mg |
| silica, magnesium stearate) in the usual amounts | |

The amounts of further adjuvants used are known to those skilled in the art from their basic knowledge, and can be taken from standard references for tablet formulation, for instance from Ritschel et al., Die Tablette, Editio Cantor, Aulendorf, 2^{nd} ed., 2002.

Properties of the mixture that is ready to be pressed, and of the tablets:

| Compressibility and flowability: | good |
|---|---|
| Mean hardness: | 91 N |
| Abrasion: | <0.1% |
| | (determined according to Ph. Eur.) |
| Disintegration time: | 68 s (determined according to Ph. Eur.) |
| Release: | 88% after 15 min |
| Content of active ingredient: | 2.53% tibolone |
| Content uniformity: | meets Ph. Eur. |
| Sum of all impurities | |
| (referred to the tibolone peak): | 0.11% |

The tablets thus obtained can be provided with a coating where applicable.

### Example No. 2

### Preparation of Tablets with Amorphous Tibolone, Formula 2 (According to the Invention)

To a solution of 1 g crystalline tibolone in 18 ml acetone/ethanol (3:1), 2 g poly-methacrylate (Eudragit® E) are added and dissolved under gentle heating (30 to 40° C.). The transparent solution is freeze-dried so as to obtain a fine powder containing active ingredients in which amorphous tibolone is present (in the following called “tibolone powder”). The tibolone powder obtained is mixed with pharmaceutical adjuvants according to the following formula, and compressed to tablets:

| | |
|---|---|
| Tibolone powder corresponding to 2.5 mg tibolone | 7.5 mg |
| Mannitol (directly compressable) | 77.5 mg |
| Adjuvants (as in Example No. 1) | 15 mg |

Properties of the mixture that is ready to be compressed, and of the tablets:

| Compressibility and flowability: | satisfactory to good |
|---|---|
| Mean hardness: | 101 N |
| Abrasion: | 0.1% |
| | (determined according to Ph. Eur.) |
| Disintegration time: | 80 s (determined according to Ph. Eur.) |
| Release: | 92% after 15 min |
| Content of active ingredient: | 2.47% tibolone |
| Content uniformity: | meets Ph. Eur. |
| Sum of all impurities | |
| (referred to the tibolone peak): | 0.12% |

The tablets thus obtained can be provided with a coating where applicable.

### Example No. 3

### Preparation of Tablets with Amorphous Tibolone, Formula 3 (According to the Invention)

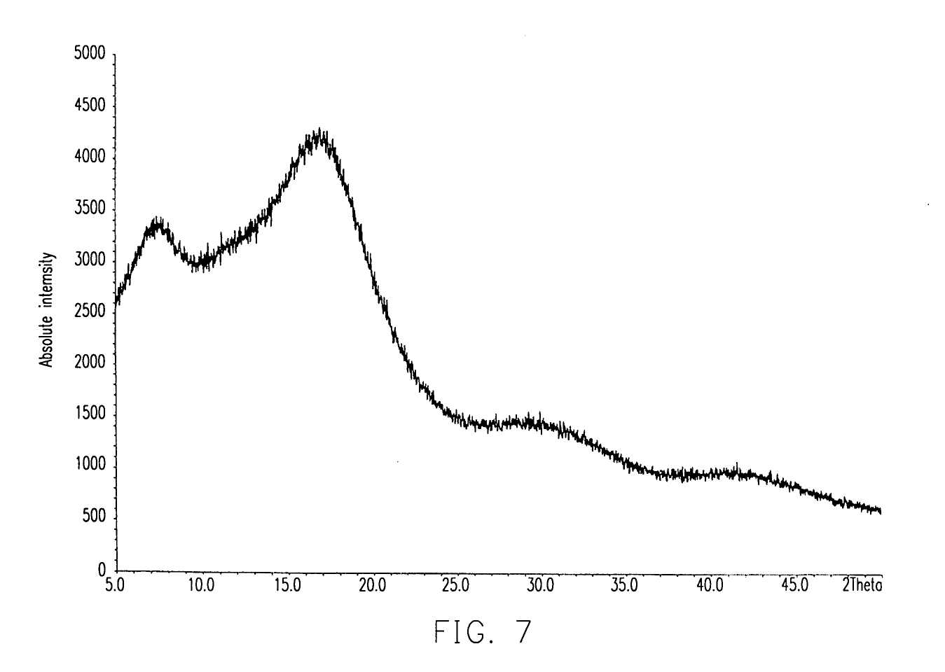

To a solution of 1 g crystalline tibolone in 20 ml acetone, 4 g polymethacrylate (Eudragit® E) are added and dissolved under gentle heating (30 to 40° C.). The transparent solution is freeze-dried so as to obtain a fine powder containing active ingredients in which amorphous tibolone is present (in the following called “tibolone powder”). The X-ray diffraction pattern of the tibolone powder obtained is presented in FIG. 7; it reveals no crystalline fractions, only amorphous fractions. The tibolone powder is mixed with pharmaceutical adjuvants according to the following formula, and compressed to tablets:

| | |
|---|---|
| Tibolone powder corresponding to 2.5 mg tibolone | 12.5 mg |
| Lactose (directly compressable) | 72.5 mg |
| Adjuvants (as in Example No. 1) | 15 mg |

Properties of the mixture that is ready to be compressed, and of the tablets:

| Compressibility and flowability: | satisfactory to good |
|---|---|
| Mean hardness: | 81 N |
| Abrasion: | 0.1% |
| | (determined according to Ph. Eur.) |
| Disintegration time: | 91 s (determined according to Ph. Eur.) |
| Release: | 84% after 15 min |
| Content of active ingredient: | 2.52% Tibolonee |
| Content uniformity: | meets Ph. Eur. |
| Sum of all impurities | |
| (referred to the tibolone peak): | 0.11% |

The tablets thus obtained can also be provided with a coating.

### Example No. 4

### Stability of Amorphous Tibolone Powder According to the Invention (According to the Invention)

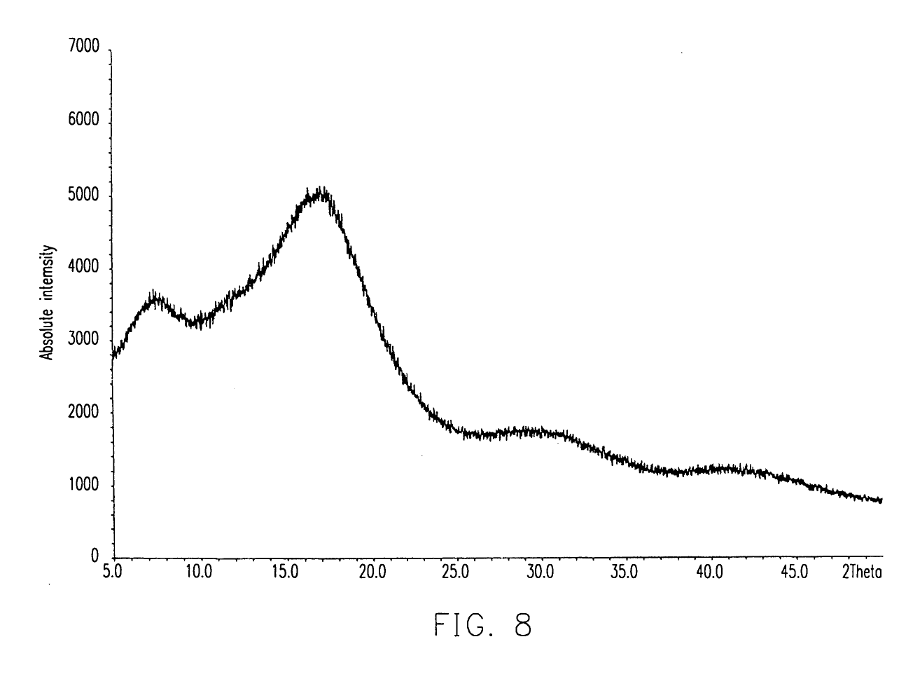

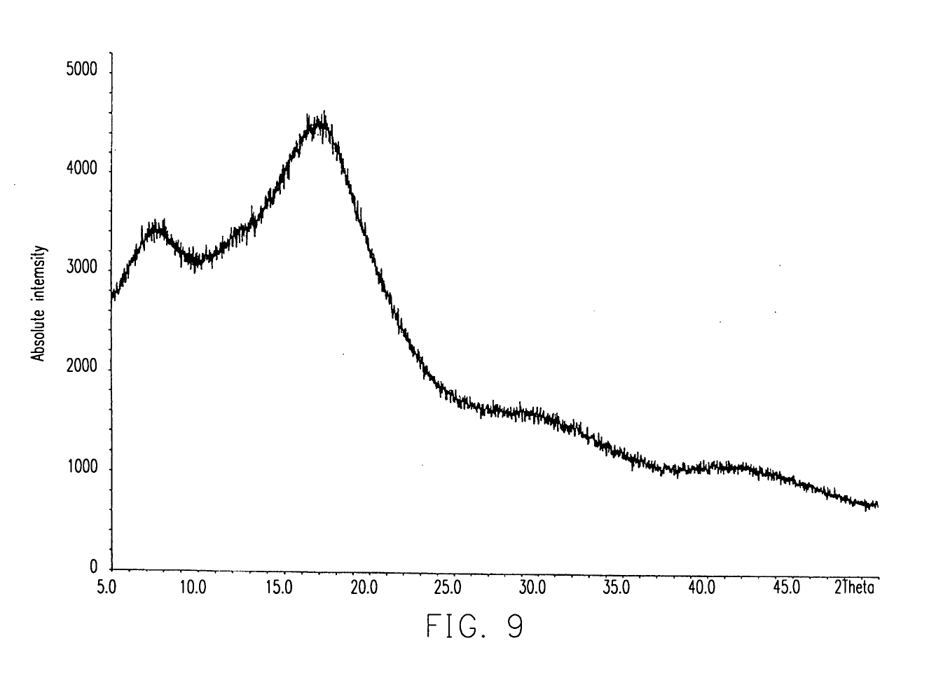

In a solution of 1 g crystalline tibolone in 15 ml acetone, 4 g polymethacrylate (Eudragit® E) are dissolved. The solvent was evaporated at 30 to 35° C. while applying a vacuum. The tibolone content of the sample was determined by HPLC, and found to be 98.8% of the nominal. The sum of all contaminants (referred to the tibolone peak) was 0.16%. The X-ray diffraction pattern of the sample recorded immediately after preparation is presented in FIG. 8, and reveals no crystalline fractions, only amorphous fractions. Storage of the sample during three months at 30° C. produces no change in morphology, that is, in the polymorphous structures (see FIG. 9); the content of active ingredient was determined to be 99.0 wt %, the sum of all contaminants was 0.29 wt %.

What is claimed is:

1. A process for the preparation of powders containing active ingredients comprising tibolone in which tibolone is present in amorphous form, comprising:
starting from a solution of tibolone in at least one organic solvent with a total water content of the solvent of no more than 15%;
dissolving in the solution of tibolone wholly or in part, a carrier material selected from the group of acrylic polymers; and
removing the solvent.

2. The process according to claim 1, wherein the total water content of the solvent is no more than 5%.

3. The process according to claim 1, in which tibolone is present in an amorphous form that is stable in storage.

4. The process according to claim 1, wherein the carrier material is selected from the group of pharmaceutical polymethacrylates consisting of methacrylic acid copolymers, aminoalkyl methacrylate copolymers, methacrylic acid ester copolymers, ammonioalkyl methacrylate copolymers.

5. The process according to claim 1, wherein a ratio of the active ingredients to the carrier material is set in the range from 1:0.1 to 1:10.

6. The process according to claim 5, wherein the ratio of the active ingredients to the carrier material is set in the range from 1:0.5 to 1:5.

7. The process according to claim 1, wherein organic solvents with a total water content of no more than 15% by volume are used alone or in mixtures as the solvent, the organic solvents being selected from the group of lower alkanols with one to four carbon atoms, the group of ethers, the esters, and the group of aliphatic ketones and their mixtures.

8. The process according to claim 7, wherein a solvent from the group consisting of methanol, ethanol, isopropanol, n-propanol, acetone, ethyl acetate, methyl ethyl ketone, methyl tert-butyl ether, dichloromethane, petroleum ether, hexane, an acetone-water mixture, an ethyl acetate-hexane mixture, a mixture of dichloromethane and ethyl acetate is employed as a solvent.

9. The process according to claim 8, wherein the solvent is kept under inert gas.

10. The process according to claim 8, wherein the solvent contains antioxidant and/or basic additives.

11. The process according to claim 1, wherein a solution of the active ingredient is employed which is obtained in the course of tibolone synthesis, and the carrier material is dissolved in it wholly or in part.

12. Powders containing active ingredients comprising tibolone in which tibolone is present in amorphous form, wherein the powders are prepared by the process according to claim 1.

13. A pharmaceutical formulation, comprising:
powders that contain pharmaceutically acceptable adjuvants and at least one active ingredient comprising tibolone in which tibolone is present in amorphous form.

14. The pharmaceutical formulation according to claim 13, wherein the formulation exists in the form of tablets and granules that are prepared with pharmaceutically acceptable adjuvants.

15. The pharmaceutical formulation according to claim 13, in the formulation is made, by direct compressing, into tablets that rapidly release the active ingredient.

16. The pharmaceutical formulation according to claim 13, wherein a binder is included in the pharmaceutical formulation and takes a portion up to 20% (m/m).

17. The pharmaceutical preparation according to claim 13, wherein fillers and adjuvants are included in the pharmaceutical formulation and together take a portion between 20 and 99% by weight.

18. The pharmaceutical preparation according to claim 17, wherein the fillers and the adjuvants together take a portion between 50 and 99% by weight.

19. Use of amorphous tibolone, obtainable by the process according to claim 1, in a formulation that is morphologically stable in storage, in oral pharmaceutical formulations.
